(19) United States
(12) Patent Application Publication
(10) Pub. No.: US 2004/0158323 A1
(43) Pub. Date: Aug. 12, 2004

(54) Method for adjusting protein affinity of hydrophilic polymers

(76) Inventors: James R. Lisk JR., Suwanee, GA (US); Phillip V. Robledo, Duluth, GA (US); Thomas R Paul, Westlake Village, CA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/716,394

(22) Filed: Nov. 18, 2003

Related U.S. Application Data
(60) Provisional application No. 60/427,704, filed on Nov. 20, 2002.

(51) Int. Cl. A61F 2/16; B24B 1/00

(52) U.S. Cl. 623/6.56; 451/34; 451/36; 623/901

(57) ABSTRACT

A system and method of processing an item at least partially formed of a hydrophilic material to reduce or increase the item's protein affinity. In one example, the system and method prevent or greatly reduce the formation of insoluble ionic materials in or on the item during processing, thereby reducing its affinity for binding with pertinacious materials. An example application involves reducing the protein affinity of an ocular item such as an intra ocular lens or a contact lens, thereby reducing the potential for in-eye or on-eye ossification of the item.

### CROSS-REFERENCE TO RELATED APPLICATION

This application claims the benefit of U.S. Provisional Patent Application Serial No. 60/427,704, filed Nov. 20, 2002, the entirety of which is hereby incorporated herein by reference for all purposes.

### FIELD OF THE INVENTION

The present invention relates generally to the processing of items formed of hydrophilic polymers, and more particularly to methods of adjusting the protein affinity of an item such as a body-contact medical device, for example a catheter, an intra ocular lens (“IOL”), a prosthetic or medical implant, or other device, formed of a hydrophilic polymer or containing a hydrophilic polymer component.

### BACKGROUND OF THE INVENTION

Intra ocular lenses, contact lenses and other items are commonly formed of hydrophilic polymers such as poly hydroxyethyl methyl-methacrylate (PHEMA), modified poly (methyl methacrylate) (PMMA), poly methacrylic acid PMAA), modified, poly acrylic acid (PAA), PHEMA hydrogels, modified poly(hydroxyethyl methylmethacryalte), poly vinyl pyrrolidone, poly vinyl alcohol or the like. Most frequently, these devices are formed of copolymers containing one or more of the following monomers: hydroxyethyl methyl-methacrylate, methyl methacrylate, methacrylic acid, vinyl pyrrolidone, vinyl acetate, vinyl alcohol and ethylenegylcol di(methyl-methacrylate). Optical clarity of such items is typically an important or desirable characteristic. Ocular devices such as intra ocular lenses and contact lenses sometimes experience in-eye or on-eye ossification or clouding resulting from deposition of a film of albumin or other pertinacious biomaterials on a surface of the device. The deposition rate is often accelerated in individuals with illnesses such as diabetes, which can modify the biochemistry of the eye. With contact lenses, such protein deposition may require replacing or cleaning the lens. With intra ocular lenses, such protein deposition may require surgically removing and replacing the device. Failure to correct the protein deposition may lead to decreased visual acuity or even blindness for the individual using the item.

Other body contact medical devices, such as urinary catheters, stents, in-dwelling access ports, sensors, prosthetics, artificial cartilage, implants, and the like, may be coated or otherwise be partially constructed of a hyrdrophillic polymer. Protein deposition on such devices may be a first step in the formation of a layer of living cells. Depending on the specific application, protein affinity may be desirable or undesirable. In many cases, the protein layer and subsequent layer of living cells may interfere with the operation of the device. In other cases, it may be desirable to have these cells for a protective or connective layer, as in the case of biological scaffolding.

Ocular items formed of hydrophilic materials with a higher affinity for protein are typically subject to varying degrees of in-eye ossification resulting from protein deposition. It has been discovered that certain processing operations carried out on items formed of hydrophilic materials can increase the protein affinity of the item, thereby leading to increased incidence of in-eye ossification. For example, phosphate buffers, borate salts, and the like are commonly used to control pH during wet-processing steps such as lens polishing. The lens polishing process traditionally has utilized a tumble-polishing slurry containing soda-lime glass beads and/or aluminum oxide polishing powder or the like, in a solution of surfactants and balanced saline solution (“BSS”, typically comprising water containing sodium chloride, calcium chloride, magnesium chloride, sodium citrate, hydrochloric acid, sodium hydroxide and/or other water-soluble salts). The lens is typically immersed in a saline solution such as BSS during its hydration and processing, and is also typically packaged and stored in BSS or other saline solution.

Multivalent anions from the buffer may combine with multivalent cations from the polishing slurry during the lens-polishing step, forming insoluble or sparingly soluble salts, such as calcium phosphate, calcium borate, calcium carbonate, magnesium phosphate and/or magnesium borate inside the matrix of and/or on the surface of the hydrophillic polymeric material. The presence of these insoluble ionic materials in and on a lens has been discovered to increase the attraction and bonding of protein molecules to the lens, thereby increasing protein affinity and the resultant potential for in-eye ossification.

Certain hydrophilic polymeric items contain ultraviolet light absorbers (UV absorbers), which contain functional groups that may bind or form ionic bonds with offensive (multivalent) cations. An example of such a UV absorber is 4-Methacryloxy-2-Hydroxybenzophenone (MOBP), which may be incorporated into the hydrophillic polymer in the polymer formation process. The complex of an offensive cation and UV absorber has also been discovered to increase the attraction and bonding of protein molecules to the lens.

Thus, it can be seen that needs exist for improved processing methods for items formed of hydrophilic materials. Needs also exist for a system and method of reducing and/or increasing the protein affinity of hydrophilic items relative to similar items produced using traditional processing methods and systems, and to hydrophilic items having adjusted protein affinity. It is to the provision of one or more methods, systems and items meeting these and other needs that the present invention is primarily directed.

### SUMMARY OF THE INVENTION

Example embodiments of the present invention provide improved processing methods for items formed at least in part of a hydrophilic material such as a hydrophilic polymer, a system and method of reducing or otherwise adjusting the protein affinity of hydrophilic polymeric items relative to similar items produced using traditional processing methods and systems, and hydrophilic polymeric items having reduced or otherwise adjusted protein affinity. Preferred and example embodiments of the invention provide an improved medical device such as an intra ocular lens or a contact lens, and a system and method of polishing or otherwise processing the device to eliminate or substantially reduce the presence of insoluble ionic materials in and on the device, and thereby reduce the protein affinity of the device and correspondingly reduce the potential for in-eye ossification or other protein deposit formation. Other embodiments include an improved medical device such as an intra ocular lens or a contact lens, and a system and method of polishing or otherwise processing the device to increase the presence of insoluble ionic materials in and on the device, and thereby increase the protein affinity of the device and correspondingly increase the potential for protein deposit formation when placed in biological contact with a user.

In one aspect, the invention is a method of processing an item at least partially formed of a hydrophilic material to produce a reduced protein affinity. The method preferably includes the prevention of the formation of insoluble ionic materials in or on the item during processing. In further preferred and optional embodiments, the method further includes hydrating the item in an aqueous solution free of multivalent cations (such as de-ionized water), processing the item in the presence of a buffer, and flushing the buffer from the item using an aqueous solution free of multivalent cations. In an example embodiment, the method comprises tumble-polishing of the item in a polishing slurry in the presence of a buffer such as a phosphate buffer.

In another aspect, the invention is a method of processing an item at least partially formed of a hydrophilic material to produce an increased protein affinity. The method preferably includes the formation of insoluble ionic materials in or on the item during processing. In further preferred and optional embodiments, the method further includes hydrating the item in an aqueous solution containing of multivalent cations (such as a calcium chloride solution in water), processing the item in the presence of a buffer, and flushing the buffer from the item using an aqueous solution or water. In an example embodiment, the method comprises hydrating the hydrophilic material in calcium chloride solution so that the calcium diffuses into the hydrophilic material matrix, and tumble-polishing of the item in a polishing slurry in the presence of a buffer such as a phosphate buffer.

In another aspect, the invention is a method of polishing an ocular item. The method preferably includes forming an ocular item at least partially from a hydrophilic material, hydrating the ocular item in an aqueous solution free of multivalent cations, polishing the ocular item in a polishing slurry solution comprising a buffer and a solvent based on deionized water or some other aqueous solution free of multivalent cations, and flushing the buffer from the ocular item using an aqueous solution free of multivalent cations.

In yet another aspect, the invention is a system for processing an item at least partially formed of a hydrophilic material to produce a reduced protein affinity, relative to similar items processed according to traditional means. The system preferably includes a hydrating chamber for hydrating one or more such items in an aqueous solution free of multivalent cations; a tumble-polisher containing a polishing slurry solution having a phosphate buffer and a solvent of an aqueous solution free of multivalent cations; and a flushing mechanism for removing the phosphate buffer from the item. The flushing mechanism may take the form of, for example, a flushing chamber and/or one or more spray heads for applying a rinse stream of de-ionized water or some other aqueous solution free of multivalent cations.

In still another aspect, the invention comprises a polishing slurry for polishing an ocular item. The polishing slurry preferably includes an aqueous solution free of multivalent cations, a plurality of polishing beads dispersed in the solution and a phosphate buffer. The slurry optionally also includes one or more surfactants and/or an abrasive polishing compounds.

In another aspect, the invention is a body-contact medical device item such as a hydrogel-based IOL or other ocular item, a catheter, stent, in-dwelling access port, sensor, prosthetic, artificial cartilage, implant, or the like, having a reduced protein affinity relative to similar items processed according to traditional means. The medical device is preferably at least partially formed of a hydrophilic material. For example, an ocular device according to the invention may have a generally transparent body bounded by at least one surface. The body and the surface are preferably substantially free of insoluble salts capable of binding to pertinacious substances. In a particular example embodiment, the ocular item is an intra ocular lens having at least one haptic extending from the generally transparent body. In another example embodiment, the ocular item is an intra ocular lens optic body, without haptics.

In another aspect, the invention is a body-contact medical device or other item having an increased protein affinity relative to similar items processed according to traditional means. The device may be, for example, a permanent medical implant item, which is preferably at least partially formed of a hydrophilic material, and has a body bounded by at least one surface. The body and the surface contain increased levels of insoluble salts capable of binding to pertinacious substances. In an example embodiment, an ocular item according to the invention is an intra ocular lens having at least one haptic extending from the generally transparent body. In another example embodiment, the ocular item is an intra ocular lens optic body, without haptics.

In another aspect, the invention is a body-contact medical device or other item having an increased protein affinity in selected portions of the device, relative to similar portions of items processed according to traditional means. The medical device may be a permanent implant item, which is preferably at least partially formed of a hydrophilic material, and in example embodiments has a generally transparent body bounded by at least one surface. The portions of the body and portions of the surface contain increased levels of insoluble salts capable of binding to pertinacious substances. In a particular example embodiment, the ocular item is an intra ocular lens having at least one haptic extending from a generally transparent body. The haptic is treated to increase protein affinity and thereby encourage livings cells to attach to the haptic, increasing the implant's stability in the implant location of the eye. In further embodiments of the invention, an IOL is processed to have an increased protein affinity in and around the haptics, and a decreased protein affinity in and around the lens body.

These and other aspects, features and advantages of the invention will be understood with reference to the detailed description herein, and will be realized by means of the various elements and combinations particularly pointed out in the appended claims. It is to be understood that both the foregoing general description and the following detailed description of the invention are exemplary and explanatory of preferred embodiments of the invention, and are not restrictive of the invention, as claimed.

### DETAILED DESCRIPTION OF EXAMPLE EMBODIMENTS

The present invention may be understood more readily by reference to the following detailed description of the invention. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of “about,” “approximately,” or the like, it will be understood that the particular value forms another embodiment.

In one example embodiment, the present invention is a method for processing an item at least partially formed of a hydrophilic material, to result in a reduction of the protein affinity of the item. For example, the item may take the form of an ocular item such as an intra ocular lens (IOL) or a contact lens, a urinary catheter, a stent, an in-dwelling access port, a sensor, a prosthetic, artificial cartilage, a biomedical implant, or the like. The item is preferably formed from a hydrophilic material such as hydroxyethyl methyl-methacrylate (HEMA), modified poly(methyl methacrylate) (PMMA), modified PMMA hydrogels, one or more copolymers of HEMA with methyl methacrylate, with monomeric UV-absorbers such as MOBP, with vinyl pyrrolidone, and/or other hydrophilic polymers. Alternatively, the item may be partially or fully coated with such a hydrophilic material, such as for example an item formed of a hydrophobic polymer or an acrylic polymer and having an exterior coating of hydrophilic polymer. The item may be produced by molding, lathing, casting and/or other forming or shaping process.

Instead of a saline solution such as BSS, the item is preferably hydrated in an aqueous solution free of multivalent cations, such as de-ionized water or a solution thereof, or a simple saline solution not containing calcium, magnesium, iron or other offensive multivalent cations. For example, an IOL or other anhydrous hydrogel item preferably is hydrated for about ninety minutes in a de-ionized water solution. The hydration optionally is carried out in an autoclave or otherwise maintained at an elevated temperature to increase the rate of hydration. The item is preferably tumble-polished or otherwise processed, for example according to known techniques in a container of polishing slurry containing soda-lime glass beads and/or aluminum oxide polishing powder or the like, and one or more surfactants, in a de-ionized water solution or another aqueous solution free of multivalent cations. A phosphate buffer is preferably added to control the pH during polishing and maintain an alkaline solution, preferably between about pH 7 and about pH 11. The buffer solution is preferably a mixture of monosodium phosphate and disodium phosphate provided in a concentration of about 0.018 mole phosphate/liter. As the glass polishing beads leach sodium and calcium during polishing, the solution pH increases, preferably to between about pH 10 and about pH 12, and calcium phosphate (as CaHPO₄) forms and precipitates as a hydrated solid (CaHPO₄.2H₂O). Since the calcium cation from the glass is bound in a solid precipitate, it is prevented from diffusing into or onto the lens.

The phosphate ion, however, was not prevented from diffusing into or onto the lens and therefore is preferably removed from the lens prior to exposing the lens to a source of calcium, magnesium, iron or other multivalent cation. The polished lens is removed from the polishing slurry and placed in de-ionized water to remove residual phosphates. Alternately, the residual phosphate may be removed in an aqueous solution, such as simple saline, that is free of multivalent cations. Preferably, the lens is rinsed with de-ionized water and immersed in de-ionized water for at least about ninety minutes for phosphate removal. Optionally, the lens is maintained at an elevated temperature in the de-ionized water, preferably about 120° C., as in an autoclave, to accelerate the rate of diffusion of phosphates from the lens. The lens is removed from the de-ionized water or other solution, and packaged, stored and/or subjected to further processing in BSS or other saline solution, according to standard practice. For example, the lens is preferably placed in BSS for at least about ninety minutes to equilibrate the lens hydration. The equilibration is optionally carried out at an elevated temperature, preferably about 120° C., as in an autoclave to increase the equilibration rate. The lens may then be processed further, if required, for example by drilling and inserting and anchoring one or more haptics, and packaged for storage and delivery.

In the above-described example, the method of the present invention comprises the exclusion or removal of potentially offensive multivalent cations, such as calcium, magnesium and/or iron from the processing solution. In this manner, anions from the buffer are not able to combine with such cations to form insoluble salts in or on the lens, which could attract and bind with ionic protein molecules and lead to accumulation of protein on the lens surface. In alternate embodiments of the invention, potentially offensive multivalent anions, such as phosphates, sulfates, carbonates and/or borates are excluded or removed from the buffer solution to prevent formation of insoluble salts in or on the lens. In still other embodiments, a chelating agent, such as for example EDTA (ethylene diamine tetra-acetic acid), is introduced to bind, potentially offensive cations to prevent the formation of insoluble salts in or on the lens. Still further embodiments of the invention eliminate or reduce the need for anionic buffers by substituting polishing slurry components that do not cause a significant pH rise during processing, such as polishing beads formed of borosilicate glass, low-sodium glass, zirconium silicate ceramic, and the like.

The present invention also comprises an ocular item or other hydrogel item processed according to any of the above-described methods or their equivalent. For example, one embodiment of the invention comprises an optical lens, such as an intra ocular lens or a contact lens at least partially formed of a hydrophilic material having a reduced protein affinity. For example, the lens preferably has a generally transparent body bounded by at least one surface, the body and the surface being maintained substantially free of insoluble salts and ionic materials during processing. The present invention also comprises a system for processing an ocular item according to any of the above-described methods or their equivalent. For example, one embodiment of the invention comprises a tumble-polishing system comprising a container containing a polishing slurry including a solution of de-ionized water.

While the invention has been described with reference to preferred and example embodiments, it will be understood by those skilled in the art that a number of modifications, additions and deletions are within the scope of the invention, as defined by the following claims.

What is claimed is:

1. A method of processing an item at least partially formed of a hydrophilic polymeric material to produce a reduced protein affinity, said method comprising preventing the formation of insoluble ionic materials in or on the item during processing.

2. The method of claim 1, further comprising:
hydrating the item in a solution free of multivalent cations;
processing the item in the presence of a buffer; and
flushing the buffer from the item using a solution free of multivalent cations.

3. The method of claim 2, further comprising tumble-polishing of the item in a polishing slurry in the presence of the buffer.

4. The method of claim 3, wherein the polishing slurry comprises glass polishing beads.

5. The method of claim 3, wherein the polishing slurry comprises a phosphate buffer.

6. The method of claim 5, wherein the item is processed in an alkaline aqueous solution.

7. The method of claim 3, wherein the polishing slurry comprises a borate buffer.

8. The method of claim 3, wherein the polishing slurry comprises a buffer selected from an acetate buffer, a citrate buffer, a carbonate buffer, and mixtures thereof.

9. The method of claim 3, wherein the polishing slurry comprises a buffer system of mixed anions.

10. The method of claim 2, wherein the step of flushing the buffer from the item in a solution free of multivalent cations is carried out at an elevated temperature.

11. The method of claim 2, further comprising equilibrating the item in a saline solution.

12. The method of claim 1, wherein the step of preventing the formation of insoluble ionic materials in or on the item during processing comprises the exclusion of multivalent cations from a processing solution.

13. An ocular item processed according to the method of claim 1.

14. A method of polishing an ocular item, said method comprising:
forming an ocular item at least partially from a hydrophilic material;
hydrating the ocular item in a solution free of multivalent cations;
polishing the ocular item in a polishing slurry solution comprising a buffer and a solvent free of multivalent cations; and
flushing the buffer from the ocular item using a solution free of multivalent cations.

15. The method of claim 14, wherein the polishing slurry solution comprises glass polishing beads and the buffer comprises a phosphate buffer.

16. The method of claim 15, further comprising maintaining the polishing slurry solution at a pH of at least 7.

17. The method of claim 14, further comprising equilibrating the ocular item in a balanced saline solution.

18. The method of claim 14, wherein the flushing step is carried out at an elevated temperature.

19. An ocular item polished according to the method of claim 14.

20. A system for processing an item at least partially formed of a hydrophilic material to produce a reduced protein affinity, said system comprising:
a hydrating chamber for hydrating the item in a solution free of multivalent cations;
a tumble-polisher containing a polishing slurry solution comprising a phosphate buffer and a solvent free of multivalent cations; and
a flushing mechanism for removing the phosphate buffer from the item.

21. A polishing slurry for polishing an ocular item, said polishing slurry comprising:
an aqueous solution free of multivalent cations;
a plurality of polishing elements dispersed in the aqueous solution; and
a phosphate buffer.

22. An ocular item having a reduced protein affinity, said ocular item being at least partially formed of a hydrophilic material, and comprising a generally transparent body bounded by at least one surface, said body and said surface being substantially free of insoluble salts capable of binding to pertinacious substances.

23. The ocular item of claim 22, comprising an intra ocular lens having at least one haptic extending from the generally transparent body.

24. The ocular item of claim 22, comprising a non-hydrophilic core with a coating of a hydrophilic polymer.

25. A method of processing an item at least partially formed of a hydrophilic polymeric material, containing organic aromatic structures, to produce a reduced protein affinity, said method comprising preventing the formation of complexes of multivalent cations with said aromatic structures, in or on the item during processing.

26. A method of processing an item at least partially formed of a hydrophilic polymeric material, said method comprising inducing the formation of insoluble ionic materials in or on the item during processing.

27. The method of claim 26, further comprising:
hydrating the item in a solution containing multivalent cations;
processing the item in the presence of a buffer; and
flushing the buffer from the item using an aqueous solution.

28. A method of processing an item at least partially formed of a hydrophilic polymeric material, containing organic aromatic structures, said method comprising inducing the formation of complexes of multivalent cations with said aromatic structures, in or on the item during processing.

29. An intra-ocular lens comprising a lens body portion and at least one haptic, said at least one haptic treated to have an increased protein affinity, and said lens body treated to have a decreased protein affinity.
